(19) United States
(12) Patent Application Publication
(10) Pub. No.: US 2026/0224182 A1
(43) Pub. Date: Aug. 6, 2026

(54) X-RAY COMPUTED TOMOGRAPHY APPARATUS, MEDICAL IMAGE PROCESSING APPARATUS, METHOD, AND STORAGE MEDIUM

(71) Applicant: Canon Medical Systems Corporation, Otawara-shi (JP)

(72) Inventors: Koki KAWAI, Kawasaki (JP); Kyotaro HORIO, Tokyo (JP)

(73) Assignee: Canon Medical Systems Corporation, Otawara-shi (JP)

(21) Appl. No.: 19/465,573

(22) Filed: Jan. 30, 2026

(30) Foreign Application Priority Data
Jan. 31, 2025 (JP) 2025-014883
Jan. 30, 2026 (JP) 2026-014409

(51) Int. Cl. A61B 6/00; A61B 6/03; A61B 6/46; A61B 6/50

(52) U.S. Cl. CPC A61B 6/5205; A61B 6/032; A61B 6/463; A61B 6/505

(57) ABSTRACT

An X-ray computed tomography apparatus includes processing circuitry. The processing circuitry acquires a medical image including a bone region. The processing circuitry generates a spatial distribution of certainty information of the bone region, based on the medical image. The processing circuitry sets a partial storage region for the bone region on the medical image, based on the spatial distribution. The processing circuitry determines validity of the partial storage region, based on the spatial distribution and position information of the partial storage region. The processing circuitry displays a display screen on which the medical image and the partial storage region are superimposed, the displaying including displaying the partial storage region with a first visual effect in a case where the validity is determined to be present, and displaying the partial storage region with a second visual effect in a case where the validity is determined to be absent.

### CROSS-REFERENCE TO RELATED APPLICATIONS

This application is based upon and claims the benefit of priority from Japanese Patent Application No. 2025-014883, filed Jan. 31, 2025, and No. 2026-14409, filed Jan. 30, 2026; the entire contents of all of which are incorporated herein by reference.

### FIELD

Embodiments described herein relate generally to an X-ray computed tomography apparatus, a medical image processing apparatus, a method, and a storage medium.

### BACKGROUND

There is known an application that automatically cuts out an oblique cross section along each of vertebrae from three-dimensional volume data including the spine, sets a storage region including each vertebra individually, and stores the storge region. In a case where a part of the spine has damage or deformation, it is difficult to automatically set the storage region at an appropriate position.

### BRIEF DESCRIPTION OF THE DRAWINGS

FIG. 1 is a diagram illustrating a configuration example of an X-ray computed tomography apparatus according to a first embodiment.

FIG. 2 is a diagram illustrating a configuration example of a medical image processing apparatus according to the first embodiment.

FIG. 3 is a diagram exemplarily illustrating a flow of a storing process of a medical image according to the first embodiment.

FIG. 4 is a diagram schematically illustrating a flow of calculation of a first center point and a second center point according to the first embodiment.

FIG. 5 is a diagram schematically illustrating a flow of setting of a partial storage region according to the first embodiment.

FIG. 6 is a diagram illustrating a display screen displaying the validity of the partial storage region according to the first embodiment.

FIG. 7 is a diagram illustrating editing of a partial storage region according to Modification 1.

FIG. 8 is a diagram exemplarily illustrating a state before editing of a partial storage region according to Modification 4.

FIG. 9 is a diagram exemplarily illustrating an initial position of the partial storage region according to Modification 4.

FIG. 10 is a diagram exemplarily illustrating a flow of a storing process of a medical image according to a second embodiment.

FIG. 11 is a diagram representing certainty information and position information relating to a vertebra according to the second embodiment.

FIG. 12 is a diagram illustrating a display screen displaying related information according to the second embodiment.

### DETAILED DESCRIPTION

A medical image processing apparatus according to one embodiment includes an acquisition unit, a generation unit, a setting unit, a determination unit, and a display unit. The acquisition unit acquires a medical image including a bone region. The generation unit generates a spatial distribution of certainty information of the bone region, based on the medical image. The setting unit sets a partial storage region for the bone region on the medical image, based on the spatial distribution. The determination unit determines validity of the partial storage region, based on the spatial distribution and position information of the partial storage region. The display unit displays a display screen on which the medical image and the partial storage region are superimposed, the display unit displaying the partial storage region with a first visual effect in a case where the validity is determined to be present, and displaying the partial storage region with a second visual effect different from the first visual effect in a case where the validity is determined to be absent.

### First Embodiment

Hereinafter, referring to the accompanying drawings, embodiments of an X-ray computed tomography apparatus, a medical image processing apparatus, a method, and a program are described in detail.

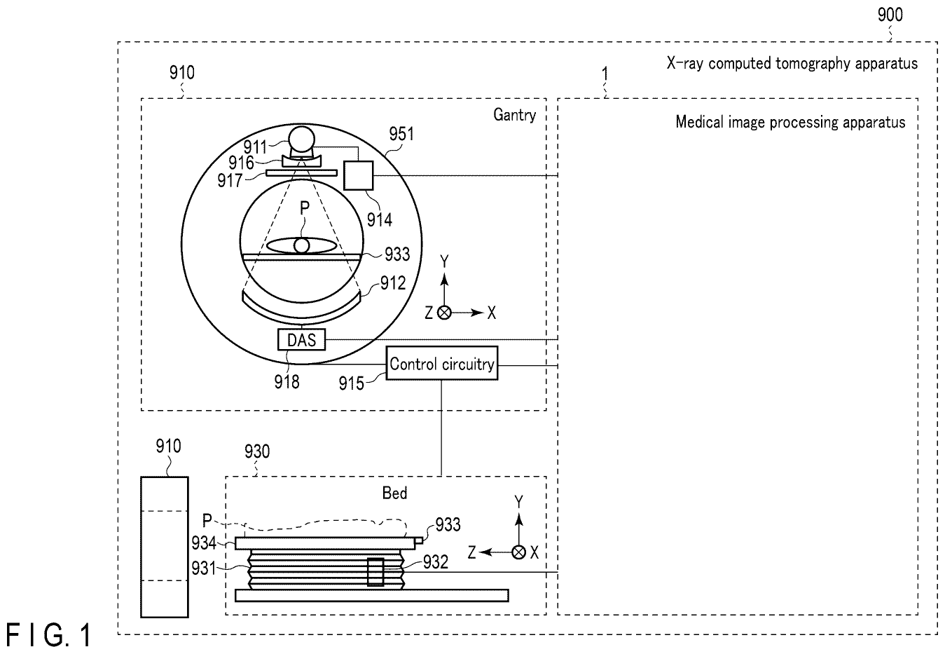

FIG. 1 is a diagram illustrating a configuration of an X-ray computed tomography apparatus 900 according to a first embodiment. The X-ray computed tomography apparatus 900 radiates X-rays from an X-ray tube 911 to a subject P, and the radiated X-rays are detected by an X-ray detector 912. Based on an output from the X-ray detector 912, the X-ray computed tomography apparatus 900 generates a CT image relating to the subject P.

As illustrated in FIG. 1, the X-ray computed tomography apparatus 900 includes a gantry 910, a bed 930 and a medical image processing apparatus 1. Note that FIG. 1 illustrates gantries 910 at a plurality of locations for the convenience of explanation, but the number of gantries 910 that are mounted in the X-ray computed tomography apparatus 900 may be one, or two or more. The gantry 910 is a scan device having a configuration for performing X-ray CT imaging on the subject P. The bed 930 is a carrier device on which the subject P that is the target of X-ray CT imaging is placed and which regulates the position of the subject P. The medical image processing apparatus 1 is, for example, a computer that controls the gantry 910. For example, the gantry 910 and the bed 930 are installed in a CT examination room, and the medical image processing apparatus 1 is installed in a control room adjacent to the CT examination room. The gantry 910, the bed 930, and the medical image processing apparatus 1 are communicably connected to one another wirelessly or by wire. Note that the medical image processing apparatus 1 need not necessarily be installed in the control room. For example, the medical image processing apparatus 1 may be installed together with the gantry 910 and the bed 930 in the same room. Alternatively, the medical image processing apparatus 1 may be incorporated into the gantry 910.

As illustrated in FIG. 1, the gantry 910 includes an X-ray tube 911, an X-ray detector 912, a rotational frame 951, an X-ray high-voltage device 914, control circuitry 915, a wedge 916, a collimator 917, and data acquisition circuitry (data acquisition system: DAS) 918.

The X-ray tube 911 emits X-rays to the subject P. Specifically, the X-ray tube 911 includes a cathode that generates thermoelectrons, an anode that generates X-rays by receiving the thermoelectrons travelling from the cathode, and a vacuum tube that holds the cathode and the anode. The X-ray tube 911 is connected to the X-ray high-voltage device 914 via a high voltage cable. The X-ray high-voltage device 914 applies a tube voltage between the cathode and the anode. Thermoelectrons travel from the cathode to the anode upon application of the tube voltage. Tube current flows as thermoelectrons travel from the cathode to the anode. By the application of high voltage from the X-ray high-voltage device 914 and the supply of filament current, thermoelectrons travel from the cathode (filament) toward the anode (target), and X-rays are generated through collision of the thermoelectrons with the anode. For example, the X-ray tube 911 may be a rotating-anode-type X-ray tube that generates X-rays by radiating thermoelectrons to a rotating anode.

The X-ray detector 912 detects the X-rays that have been emitted from the X-ray tube 911 and have passed through the subject P, and outputs an electric signal corresponding to the detected X-ray dose to the data acquisition circuitry 918. The X-ray detector 912 has a structure in which a plurality of X-ray detection element rows are aligned in a slice direction (row direction), each of the X-ray detection element rows including a plurality of X-ray detection elements aligned in a channel direction. The X-ray detector 912 is, for example, an indirect conversion-type detector including a grid, a scintillator array, and an optical sensor array. The scintillator array includes a plurality of scintillators. The scintillator outputs an amount of light corresponding to an amount of incident X-rays. The grid is arranged on the X-ray incident surface side of the scintillator array, and includes an X-ray shielding plate that absorbs scattered X-rays. The grid may be referred to as a “collimator (one-dimensional collimator or two-dimensional collimator)”. The optical sensor array converts the light to an electric signal corresponding to the amount of light output from the scintillator. For example, a photodiode is used as the optical sensor. Note that the X-ray detector 912 may be a direct conversion-type detector.

The rotational frame 951 is an annular frame that supports the X-ray tube 911 and the X-ray detector 912 rotatably about the rotational axis (Z axis). In the rotational frame 951, holes or recesses (hereinafter “front surface”) for attaching various devices such as the X-ray tube 911, X-ray detector 912, high-voltage generator and data acquisition circuitry 918 are formed. Specifically, the rotational frame 951 supports the X-ray tube 911 and the X-ray detector 912 in such a manner that the X-ray tube 911 and the X-ray detector 912 face each other. The rotational frame 951 is supported by a stationary frame (not illustrated) so as to be rotatable around the rotational axis. By the rotation of the rotational frame 951 around the rotational axis by the control circuitry 915, the X-ray tube 911 and the X-ray detector 912 rotate around the rotational axis. The rotational frame 951 receives driving force from a driving mechanism of the control circuitry 915, and rotates at a predetermined speed around the rational axis. A field of view (FOV) of an image is set in an opening of the rotational frame 951.

Note that in the present embodiment, a rotational axis of the rotational frame 951 in a non-tilt state, or a longitudinal direction of a top plate 933 of the bed 930, is defined as a Z-axis direction, an axial direction that is perpendicular to the Z-axis direction and is horizontal to the floor surface is defined as an X-axis direction, and an axial direction perpendicular to the Z-axis direction and is perpendicular to the floor surface is defined as a Y-axis direction.

The X-ray high-voltage device 914 includes a high-voltage generator and an X-ray controller. The high-voltage generator includes electric circuitry, such as a transformer and a rectifier, and generates high voltage to be applied to the X-ray tube 911, and filament current to be supplied to the X-ray tube 911. The X-ray controller controls output voltage in accordance with the X-rays emitted by the X-ray tube 911. The high-voltage generator may adopt a transformer system or an inverter system. The X-ray high-voltage device 914 may be provided to the rotational frame 951 in the gantry 910 or provided to the stationary frame (not illustrated) in the gantry 910.

The wedge 916 adjusts the dose of X-rays emitted to the subject P. Specifically, the wedge 916 attenuates the X-rays so that the dose of X-rays emitted from the X-ray tube 911 to the subject P exhibits a predetermined distribution. For example, a metal plate made of aluminum or the like, such as a wedge filter or a bow-tie filter, is used as the wedge 916.

The collimator 917 limits the range of radiation of X-rays that have passed through the wedge 916. The collimator 917 slidably supports a plurality of lead plates that shield X-rays and adjusts the shape of slits formed by the lead plates. The collimator 917 may be referred to as an X-ray diaphragm.

The data acquisition circuitry 918 reads from the X-ray detector 912 electric signals corresponding to the dose of X-rays detected by the X-ray detector 912. The data acquisition circuitry 918 amplifies the read electric signals and integrates the electric signals during a view period, thereby acquiring projection data with a digital value corresponding to the dose of X-rays during the view period. The data acquisition circuitry 918 is implemented by, for example, an application specific integrated circuit (ASIC) equipped with a circuit element capable of generating projection data. The digital data is transmitted to the medical image processing apparatus 1 via a non-contact data transmitter or the like.

The control circuitry 915 controls the X-ray high-voltage device 914 or the data acquisition circuitry 918 to perform X-ray CT imaging in accordance with an imaging control function 120 of processing circuitry 11 of the medical image processing apparatus 1. The control circuitry 915 includes processing circuitry including a central processing unit (CPU), a micro processing unit (MPU), or the like, and a drive mechanism such as a motor or an actuator. The processing circuitry includes, as hardware resources, a processor such as a CPU or the like and a memory such as a read-only memory (ROM), a random access memory (RAM), or the like. In addition, the control circuitry 915 may be realized by an ASIC or a field programmable gate array (FPGA). The control circuitry 915 may also be realized by a complex programmable logic device (CPLD) or a simple programmable logic device (SPLD). The control circuitry 915 has a function of executing operational control of the gantry 910 and the bed 930 by receiving an input signal from an input interface 41 (to be described later) attached to the medical image processing apparatus 1 or the gantry 910. For example, the control circuitry 915 receives input signals and executes control to rotate the rotational frame 951, control to tilt the gantry 910, and control to move the bed 930 and the top plate 933. Note that the control to tilt the gantry 910 is implemented by the control circuitry 915 rotating the rotational frame 951 around an axis parallel to the X-axis direction, based on inclination angle (tilt angle) information that is input by the input interface attached to the gantry 910. The control circuitry 915 may be provided to the gantry 910 or the medical image processing apparatus 1.

The bed 930 includes a base 931, a support frame 932, the top plate 933, and a bed driving device 934. The base 931 is installed on the floor. The base 931 is a housing that supports the support frame 932 movably in a perpendicular direction (Y-axis direction) to the floor. The support frame 932 is a frame provided on top of the base 931. The support frame 932 supports the top plate 933 slidably along the rotational axis (Z axis). The top plate 933 is a plate with flexibility, on which the subject P is placed.

The bed driving device 934 is housed in the housing of the bed 930. The bed driving device 934 is a motor or actuator that generates driving force to move the support frame 932 and the top plate 933, on which the subject P is placed. The bed driving device 934 operates in accordance with the control executed by the medical image processing apparatus 1, and the like.

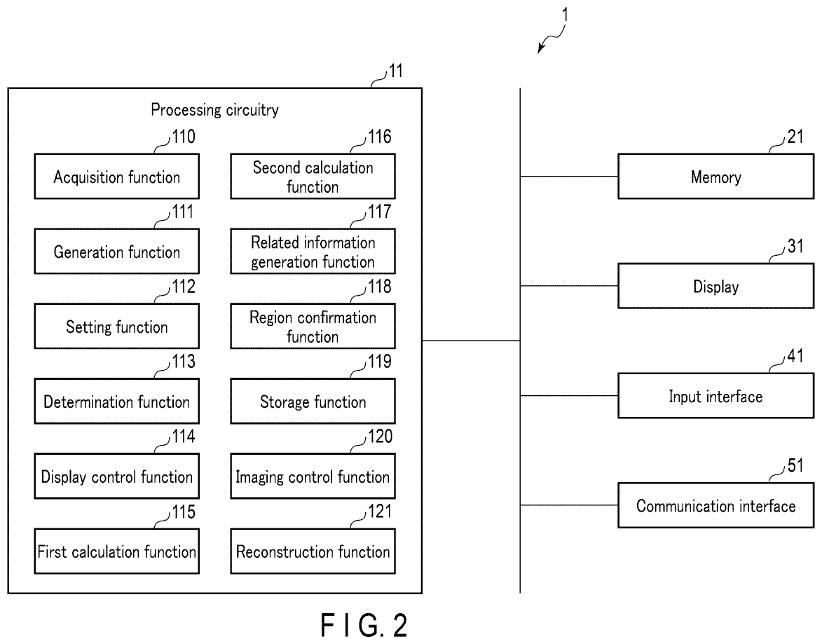

FIG. 2 is a diagram illustrating a configuration example of the medical image processing apparatus 1 according to the first embodiment. As illustrated in FIG. 2, the medical image processing apparatus 1 includes processing circuitry 11, a memory 21, a display 31, an input interface 41, and a communication interface 51. Data communication between the processing circuitry 11, memory 21, display 31, input interface 41 and communication interface 51 is executed via a bus.

The memory 21 is a storage device, such as a hard disk drive (HDD), a solid state drive (SSD), or an integrated circuit storage device, which stores various types of information. The memory 21 stores, for example, projection data and reconstruction image data. Aside from an HDD, an SSD, or the like, the memory 21 may be a portable storage medium such as a compact disc (CD), a digital versatile disc (DVD), or a flash memory. The memory 21 may be a drive device that reads and writes various types of information from and in, for example, a semiconductor memory device such as a flash memory or a RAM. Besides, the storage area of the memory 21 may be in the medical image processing apparatus 1 or in an external storage device connected via a network.

The display 31 displays various kinds of information. For example, the display 31 outputs a medical image (CT image and/or MR image) generated by the processing circuitry, and a graphical user interface (GUI) for receiving various operations from a user. Various types of freely selected displays can be used as appropriate as the display 31. For example, a liquid crystal display (LCD), a cathode ray tube (CRT) display, an organic electroluminescence display (OELD), or a plasma display can be used as the display 31.

The input interface 41 is an interface for receiving various input operations from the user, converting the received input operations into electric signals, and outputting the electric signals to the processing circuitry 11. For example, a mouse, a keyboard, a trackball, a switch, a button, a joystick, a touch pad, a touch panel display, or the like can be suitably used as the input interface 41. In the embodiment, the input interface 41 does not necessarily include physical operation components such as a mouse, a keyboard, a trackball, a switch, a button, a joystick, a touch pad, and a touch panel display. Examples of the input interface 41 include processing circuitry for electric signals, which receives an electric signal corresponding to an input operation from an external input device provided separately from its own apparatus, and outputs this electric signal to the processing circuitry 11.

The communication interface 51 is an interface for data communication between the medical image processing apparatus 1 and another computer and/or a sensor. For example, the communication interface 51 transmits and receives, via a network, medical images between the medical image processing apparatus 1 and sensors of computers or the like, such as a picture archiving and communication system (PACS), a hospital information system (HIS), and/or a radiology information system (RIS).

The processing circuitry 11 controls the overall operation of the medical image processing apparatus 1 in accordance with an electric signal of an input operation that is output from the input interface 41. For example, the processing circuitry 11 includes, as hardware resources, a processor such as a CPU, and a memory such as a ROM and a RAM. The processing circuitry 11 executes an acquisition function 110, a generation function 111, a setting function 112, a determination function 113, a display control function 114, a first calculation function 115, a second calculation function 116, a related information generation function 117, a region confirmation function 118, a storage function 119, a imaging control function 120, and a reconstruction function 121, by a processor that executes a program loaded in the memory. The functions 110 to 121 are not necessarily implemented by single processing circuitry. The processing circuitry may be constituted by combining a plurality of independent processors, and the functions 110 to 121 may be implemented by the respective processors executing programs.

By implementing the acquisition function 110, the processing circuitry 11 acquires various images. The processing circuitry 11 acquires, for example, a medical image including a bone region. The medical image may be a CT image or an MR image. In addition, although the medical image is assumed to be three-dimensional image data (volume data), the medical image may be two-dimensional image data.

By implementing the generation function 111, the processing circuitry 11 generates a spatial distribution of certainty information of a bone region, based on the medical image. The bone region is a region assumed to be a bone in the subject P. The certainty information is information, such as a numerical value or a sign, indicating the certainty of a part of the subject P imaged at each pixel.

By implementing the setting function 112, the processing circuitry 11 sets a partial storage region on the medical image, based on the spatial distribution. In addition, by implementing the setting function 112, the processing circuitry 11 sets a partial storage region on the medical image, based on the spatial distribution and a first center point. The partial storage region is a region for cutting out and storing a portion of the medical image.

By implementing the determination function 113, the processing circuitry 11 determines validity of the partial storage region, based on the spatial distribution and the position information of the partial storage region. The validity is determined with respect to the position and/or size of the partial storage region that is set on a target bone region. The target bone region is a set of spatially continuous pixels, in which the certainty information exceeds a predetermined threshold on the spatial distribution of the certainty information of the bone region. The target bone region is, for example, one vertebra region included in the spine region.

By implementing the display control function 114, the processing circuitry 11 displays a display screen on which the partial storage region is superimposed on the medical image. In addition, in a case where the validity is determined to be present, the processing circuitry 11 displays the partial storage region with a first visual effect, and in a case where the validity is determined to be absent, the processing circuitry 11 displays the partial storage region with a second visual effect that is different from the first visual effect. The first visual effect and/or the second visual effect is, for example, an effect that displays the partial storage region with a predetermined color. However, the first visual effect and/or the second visual effect is not limited to the displaying with the predetermined color. For example, the visual effect may be a pattern, flickering, transparency, and/or a kind of a frame line with respect to the partial storage region.

By implementing the first calculation function 115, the processing circuitry 11 calculates a first center point as a center point of a region in which the certainty information exceeds a first threshold in the spatial distribution. The first center point is used, for example, for the setting of the partial storage region and for the determination of the validity of the partial storage region.

By implementing the second calculation function 116, the processing circuitry 11 calculates a second center point as a center point of a region in which the certainty information exceeds a second threshold in the spatial distribution, the second threshold being less than the first threshold. The second center point is used, for example, for the determination of the validity of the partial storage region.

By implementing the related information generation function 117, the processing circuitry 11 generates related information that relates to an anatomical part included in the partial storage region, based on the partial storage region determined to have validity.

By implementing the region confirmation function 118, the processing circuitry 11 confirms the partial storage region in accordance with the user's instruction.

By implementing the storage function 119, the processing circuitry 11 cuts out and stores partial data of the medical image, the partial data corresponding to the partial storage region. The partial data is stored, for example, in the memory 21.

In the imaging control function 120, the processing circuitry 11 controls the X-ray high-voltage device 914, the control circuitry 915, and the DAS 918. In the imaging control function 120, the processing circuitry 11 controls the bed 930, for example, in order to dispose a mounting tool, which is provided on the top plate 933, below the scanner.

In the reconstruction function 121, the processing circuitry 11 generates a CT image or the like, based on the projection data output from the DAS 918. In addition, the processing circuitry 11 may convert a CT image to a given cross-sectional image or a rendering image in a given direction of a visual point. The conversion is performed based on an input operation received from an operator through the input interface 41. For example, the processing circuitry 11 subjects the CT image to three-dimensional image processing such as volume rendering, surface volume rendering, pixel value projection processing, multi-planar reconstruction (MPR) processing, curved MPR (CPR) processing, or the like, and generates rendering image data in a given direction of a visual point.

Hereinafter, the medical image processing apparatus 1 according to the first embodiment is described in detail.

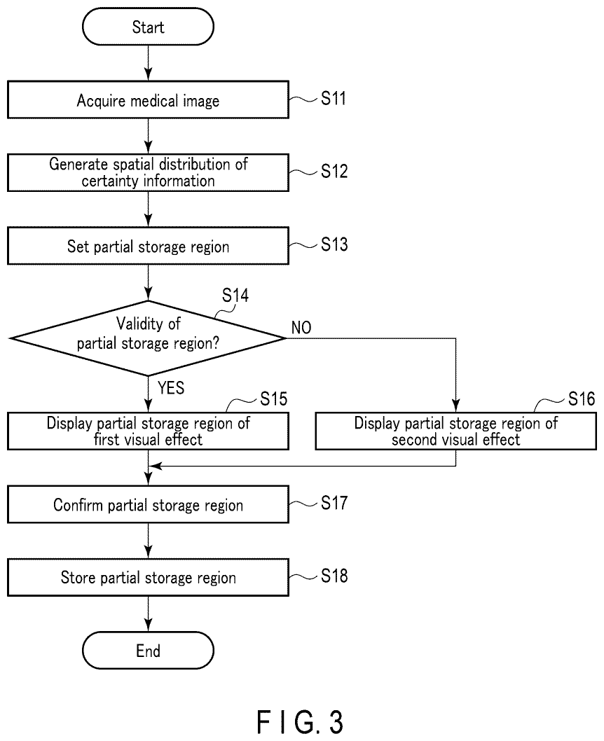

FIG. 3 is a diagram illustrating a processing procedure of a storing process of a medical image according to the first embodiment. As illustrated in FIG. 3, by implementing the acquisition function 110, the processing circuitry 11 acquires a medical image including a bone region (step S11). The processing circuitry 11 acquires the medical image, for example, from the medical image processing apparatus 1 and/or the PACS or the like via the communication interface 51. The bone region included in the medical image is, for example, the spine region of the subject P. Note that the bone region included in the medical image is not limited to the spine region of the subject P. For example, the bone region included in the medical image may be a rib region or the like of the subject P.

If step S11 is executed, the processing circuitry 11 generates, by implementing the generation function 111, a spatial distribution of certainty information of the bone region, based on the medical image acquired in step S11 (step S12). Hereinafter, the spatial distribution of the certainty information is referred to as “certainty map”. For example, by inputting the medical image into a machine learning model, the processing circuitry 11 generates the certainty map as an intermediate output that is an output of an image in which the bone region included in the medical image is segmented. To be more specific, the machine learning model is a machine learning model that is trained to generate an image in which a bone region is segmented from a medical image, by using, as learning samples, medical images collected from a plurality of subjects, and images in which bone regions are segmented from the medical images, and the machine learning model is a machine learning model that uses a certainty map as an intermediate output. As the machine learning model, for example, use may be made of a convolutional neural network based on the U-net that is generally used in segmentation. In one example, the certainty map of the bone region is generated as a heat map indicating the certainty information relating to the segmentation of the bone region. Hereinafter, the bone region is assumed to be the spine region, but may be a rib region or the like.

If step S12 is executed, the processing circuitry 11 sets, by implementing the setting function 112, a partial storage region on the medical image acquired in step S11, based on the certainty map generated in step S12 (step S13). The processing circuitry 11 sets the size and/or position of the partial storage region, based on the certainty map of the bone region. The processing circuitry 11 sets, for example, a partial storage region for each of vertebra regions on the certainty map of the spine region.

In one example, the processing circuitry 11 calculates, by implementing the first calculation function 115, a first center point as a center point of a region in which the certainty information exceeds a first threshold in the certainty map. The processing circuitry 11 calculates, for example, the first center point from each of the vertebra regions. By implementing the setting function 112, the processing circuitry 11 sets a center point of the partial storage region at the calculated first center point. As the first threshold, a predetermined value may be set. The processing circuitry 11 may calculate a center line of the spine, and may set the partial storage region as a rectangular parallelepiped that has a center at the first center point and has a plane perpendicular to the center line.

In addition, for example, in order to determine the validity of the partial storage region, the processing circuitry 11 calculates a center point of each of the vertebra regions, based on a different standard from the first threshold. In one example, by implementing the second calculation function 116, the processing circuitry 11 calculates a second center point as a center point of a region in which the certainty information exceeds a second threshold in the certainty map, the second threshold being less than the first threshold.

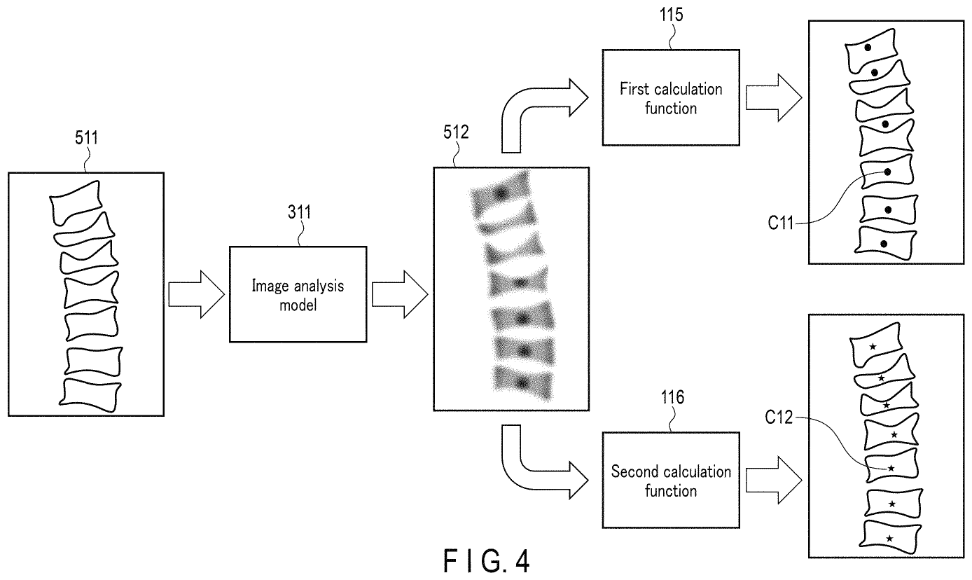

FIG. 4 is a diagram exemplarily illustrating a flow of calculation of a first center point C11 and a second center point C12. As illustrated in FIG. 4, an image analysis model 311 is a machine learning model to which a medical image 511 is input, and which outputs a certainty map 512. The certainty map 512, which is illustrated for the purpose of description, is a heat map in which values of the certainty map of the bone region are displayed by a color scale. The spine region is included in the medical image 511 illustrated in FIG. 4. In the certainty map 512 illustrated in FIG. 4, each of vertebra regions of the spine region is extracted. However, the image analysis model 311 is not limited to the machine learning model that outputs the certainty map 512 as an intermediate output. For example, the image analysis model 311 may be a machine learning model that is trained to output the certainty map 512. Besides, the certainty map 512 is not limited to the heat map. For example, the certainty map 512 may be an array in which values of spatial coordinates and certainties are stored.

Further, as illustrated in FIG. 4, the generated certainty map 512 is input to the first calculation function 115 and the second calculation function 116. By implementing the first calculation function 115, the processing circuitry 11 calculates the first center point C11, based on the input certainty map 512. The first calculation function 115 calculates, as the first center point C11, a center point of a region having a certainty exceeding a first threshold in the generated certainty map 512. The first center point C11 is, for example, a center point of a bone region in volume data. For example, in a case where the medical image 511 includes an abnormal spine region such as scoliosis, fracture of a centrum, fixture of a vertebra by a bolt, shading on an image, or vertebral anomaly, the accuracy, with which the processing circuitry 11 calculates the first center point C11 as the center point of each of the vertebra regions, lowers. As illustrated in FIG. 4, there is a case where the first center point C11 is set at a position that is not on the vertebra region. The second calculation function 116 calculates the second center point C12, based on the input certainty map 512. The second calculation function 116 calculates, as the second center point C12, a center point of a region having a certainty exceeding a second threshold in the generated certainty map 512. The second threshold has a smaller value than the first threshold. The second center point C12 is, for example, a center point of a bone region in volume data. With the second threshold being set at a smaller value than the first threshold, the certainty becomes lower than in the case of the first threshold, and thus the processing circuitry 11 can calculate the second center point from a region that is not the calculation target in the first calculation function 115.

Note that the first calculation function 115 and/or the second calculation function 116 may be implemented by the image analysis model 311 described with reference to FIG. 4. By implementing the first calculation function 115, the processing circuitry 11 may calculate a center point of an intervertebral disk region of the spine region. Further, although the first center point C11 and the second center point C12 are illustrated for the purpose of description, the first center point C11 and the second center point C12 may not be output as images. For example, only coordinates may be calculated as the first center point C11 and the second center point C12. Besides, the calculation of the first center point C11 and the second center point C12 is not necessarily limited to the calculation by the same input.

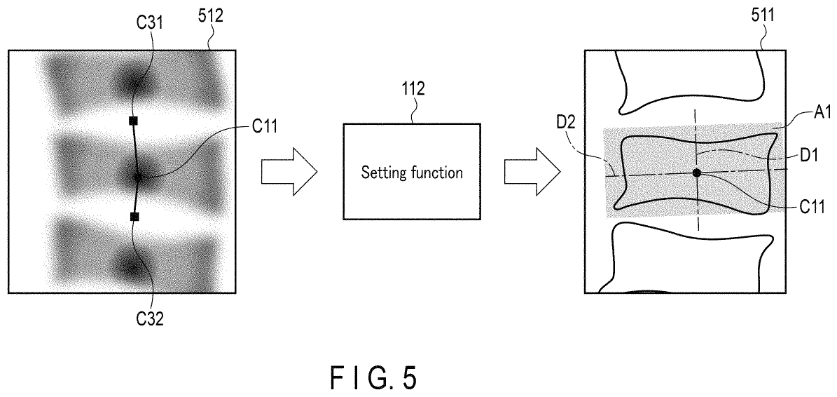

FIG. 5 is a diagram exemplarily illustrating a flow of setting of a partial storage region A1. As illustrated in FIG. 5, by implementing the setting function 112, a center point C31 and a center point C32 of two intervertebral disk regions neighboring the calculated first center point C11 and the vertebral region including the calculated first center point C11 are input to the processing circuitry 11, and the processing circuitry 11 sets the partial storage region A1 for the medical image 511. As illustrated in FIG. 5, the input is the first center point C11, and the center point C31 and center point C32 of the two neighboring intervertebral regions, which are calculated on the certainty map 512 of the spine region. The output is the partial storage region A1 that is set for one vertebral region of the medical image 511 of the spine region corresponding to the input. By implementing the setting function 112, the processing circuitry 11 sets the length of the partial storage region in the center-line direction of the spine region, based on the distance between the first center point C11 and the center point C31 and/or the center point C32 of the intervertebral disk region neighboring the vertebral region including the first center point C11.

The length of the partial storage region A1 in a center-line direction D1 of the spine region may be set at a predetermined value, or may be set in accordance with the user's instruction. In addition, the length may be set by the center points C31 and C32 of the neighboring intervertebral disk regions. Further, there may be a case where line segments connecting the first center point C11 and the center points C31 and/or C32 of the two intervertebral disk regions are not necessarily parallel and do not have identical distances. In this case, the length of the partial storage region centering at the first center point C11 is defined, and thus the length of the partial storage region A1 in the center-line direction D1 has a minimum value and/or a maximum value restricted by the distances from the first center point C11 to the center points C31 and/or C32 of the two intervertebral disk regions, and may be set at a value in which the sum of certainties included in the region satisfying the restriction and the density of certainties are optimized. In this case, the distance from the first center point C11 to the center point C31 of the intervertebral disk region is set to be the maximum value, and the distance from the first center point C11 to the center point C32 of the intervertebral disk region is set to be the minimum value. The length of the partial storage region A1 in the direction D2 perpendicular to the center-line direction D1 of the spine region may be set at a predetermined value, or may be set in accordance with the user's instruction. The partial storage region A1 for one vertebra region can be set by setting the length of the partial storage region A1 in the center-line direction D1 of the spine region, based on the first center point C11 and the center points C31 and/or C32 of the intervertebral disk regions.

Note that the setting of partial storage regions for a plurality of vertebra regions may be executed by repeating multiple times the above-described flow of setting. In a case where a center point of an intervertebral disk region does not exist on both sides of the center point of a vertebra of interest, such as in an endmost edge portion of a vertebra, the partial storage region may be defined by using a fixed ratio or the like from the center point of the vertebra. Also, in a case where a center point of an intervertebral disk region exists only on one side of the center point of a vertebra of interest, such as in a sacrum, the partial storage region may be defined based on the center point of the vertebra and the center point of the intervertebral disk region.

If step S13 is executed, the processing circuitry 11 determines, by implementing the determination function 113, the validity of the partial storage region that is set in step S13, based on the certainty map generated in step S12 and the position information of the partial storage region set in step S13 (step S14). In one example, the processing circuitry 11 calculates, by implementing the determination function 113, the distance between the first center point and the second center point, and determines that the validity is present if the distance from the first center point is less than a third threshold, and determines that the validity is absent if the distance from the first center point is not less than the third threshold. The distance between the first center point and the second center point is a Euclidean distance between the first center point and the second center point, which are calculated as points on the coordinates in the certainty map. The processing circuitry 11 determines, by implementing the determination function 113, that the validity is present, for example, if the difference between the third threshold and the distance between the first center point and the second center point is a non-negative value, and determines that the validity is absent if the difference is a negative value. The third threshold may be set at a predetermined value, or may be set at a freely selected value in accordance with the user's instruction. In regard to the distance between the first center point and the second center point, in a case where a plurality of second center points are calculated, the validity may be determined based on the distance between the coordinates of the first center point and the coordinates of the second center point closest to the coordinates of the first center point. In a case where a plurality of partial storage regions are set for one medical image, the processing circuitry 11 determines the validity of each of the partial storage regions.

If the validity is determined to be present in step S14 (step S14: YES), the processing circuitry 11 displays, by implementing the display control function 114, the partial storage region set in step S13, by using the first visual effect (step S15).

If the validity is determined to be absent in step S14 (step S14: NO), the processing circuitry 11 displays, by implementing the display control function 114, the partial storage region set in step S13, by using the second visual effect (step S16).

If step S15 or step S16 is executed, the processing circuitry 11 confirms, by implementing the region confirmation function 118, the partial storage region displayed in step S15 or step S16, in accordance with the user's instruction (step S17). The confirmation process of the partial storage region may be executed, for example, on the display screen that displays the partial storage region.

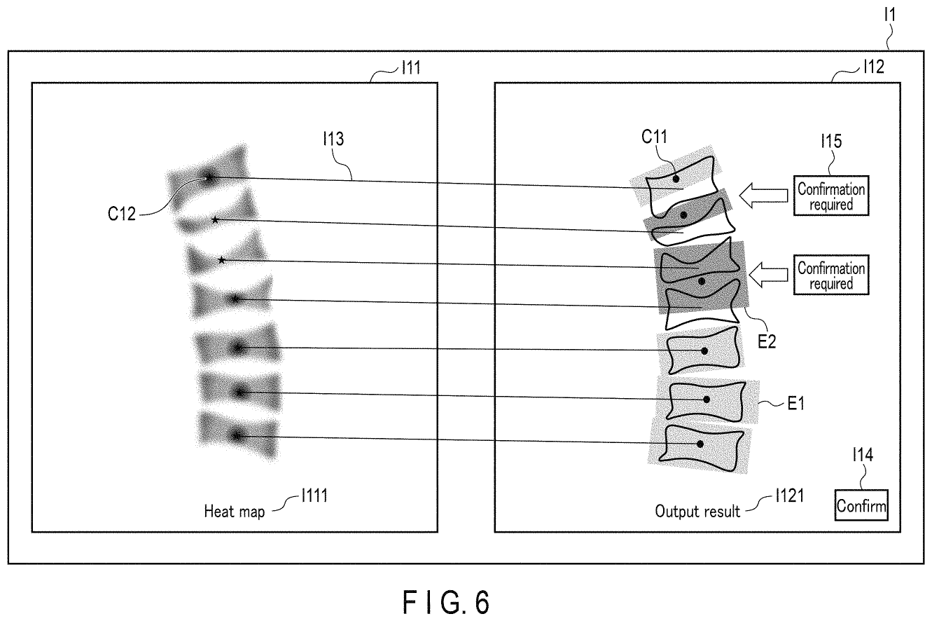

FIG. 6 is a diagram illustrating a display screen I1 displaying the validity of the partial storage region. As illustrated in FIG. 6, the display screen I1 includes a first display area I11 indicating the certainty map and the second center point C12; a second display area I12 indicating the medical image and the position information of the partial storage region; a correspondence line I13 that associates the position of the second center point C12 displayed in the first display area I11 with the position of the second center point C12 of the medical image displayed in the second display area I12; a button I14 for confirming the partial storage region; and a character string I15 for prompting the user to edit.

In the first display area I11, the second center point C12 is displayed by being superimposed on the certainty map. The certainty map is a heat map of the certainty map of the spine region. The second center point C12 is a point calculated based on the certainty map displayed in the first display area I11. In the second display area I12, the partial storage region is displayed by being superimposed on the medical image and the first center point C11. The first center point is a point calculated based on the certainty map displayed in the first display area I11. The partial storage region displayed in the second display area I12 is displayed with a first visual effect E1 or a second visual effect E2, based on the determination. In one example, the first visual effect E1 is an effect of displaying the partial storage region in yellow. The second visual effect E2 is an effect of displaying the partial storage region in red. Character strings, which can distinguish the first display area I11 and the second display area I12, may also be displayed in the first display area I11 and the second display area I12. As illustrated in FIG. 6, a character string I111, such as “Heat map”, may be displayed in the first display area I11. A character string I121, such as “Output result”, may be displayed in the second display area I12.

The correspondence line I13 is displayed in such a manner as to connect the position of the second center point C12 displayed in the first display area I11 to the position corresponding to the second center point in the medical image of the second display area I12. The button I14 for confirming the partial storage region is a button that can be selected by a mouse and/or the input interface 41 such as a touch panel. As illustrated in FIG. 6, a character string, such as “Confirm”, indicating that the partial storage region is confirmed, may be displayed on the button I14 for confirming the partial storage region.

In addition, the processing circuitry 11 displays, by implementing the display control function 114, the partial storage region on the medical image in such a manner that the partial storage region can be edited in accordance with the user's instruction. The editing is, for example, an operation including movement, enlargement/reduction, rotation or deformation of the partial storage region. In one example, by the user selecting a specific partial storage region, the display screen I1 transitions to a mode in which the selected partial storage region can be edited. In addition, the processing circuitry 11 displays, by implementing the display control function 114, the character string I15 prompting the user to edit, in regard to the partial storage region displayed with the second visual effect in the second display area I12. As illustrated in FIG. 6, as the displayed character string I15, a character string, such as “Confirmation required”, prompting the user to edit, may be displayed. In addition, a sign for indicating the partial storage region, to which the character string I15 prompting the user to edit corresponds, is displayed in the second display area I12. The sign may be an arrow or the like, which indicates the partial storage region.

Since the partial storage regions are displayed with the first visual effect and the second visual effect, the user can easily confirm the presence/absence of the validity of each of the partial storage regions, and, by extension, it is possible to reduce the time needed for examining the partial storage region that requires editing. Furthermore, by displaying the first display area and the second display area together, the user can easily confirm the basis of the determination. By the button for confirming the partial storage region, it is possible to determine whether or not to confirm the partial storage region in accordance with the user's instruction.

Note that the certainty map in the first display area and/or the medical image in the second display area may be displayed as volume data.

If step S17 is executed, the processing circuitry 11 stores, by implementing the storage function 119, a part of the medical image included in the partial storage region confirmed in step S17 (step S18). The target of storage is, for example, the entirety of the confirmed partial storage region. In one example, in the processing circuitry 11, with a partial storage region being confirmed, each partial storage region is stored. For example, the processing circuitry 11 cuts out a part of the medical image included in the confirmed partial storage region, and stores the cut-out part in the memory 21. The cut-out medical image may be stored in the memory 21 by being associated with the original medical image. A part of the medical image included in the partial storage region desired by the user can be cut out and stored. Note that the target of storage is not limited to the confirmed partial storage region. The target of storage may be the entirety of the partial storage region set by the setting function 112, or may be the entirety of the partial storage region determined to have the validity. In this case, without confirming the partial storage region, the processing circuitry 11 may automatically execute the storing process at a timing when the partial storage region is set or at a timing when the validity is determined.

If step S18 is executed, the storing process of the medical image according to the first embodiment is completed.

Note that the second center point may be calculated at any timing if the second center point is calculated at a stage prior to step S13. In addition, although the case was described in which the first center point and the second center point are used for the setting of the partial storage region, the present embodiment is applicable even if the partial storage region is set by any method. For example, without calculating the first center point, the partial storage region may be set for each of regions in which the certainty information exceeds a predetermined threshold.

Here, the first embodiment is compared with a comparative example in which the validity of the partial storage region is not determined. In the comparative example, a certainty map of a bone region is generated from a medical image by a machine learning model, and a partial storage region is set for the generated certainty map. However, in a machine learning model trained by medical images including normal bone regions, since it is difficult to accurately calculate certainty information of medical images including deformed or damaged bone regions, or shading due to a metallic corrective jig such as a bolt, there is a case where a partial storage region that is set is different from a position desired by the user. Thus, the user needs to confirm all partial storage regions that are set, and to examine validity as to whether or not to edit the set partial storage regions. Compared to the comparative example, according to the present embodiment, by determining the validity of the set partial storage region and displaying the partial storage region with different visual effects according to the determination, the user can easily confirm the validity of each partial storage region. Furthermore, the validity of the position where the partial storage region is set can be determined by comparing the center point of the partial storage region, which is set based on the certainty map generated by the machine learning model of partial storage regions, with the center point of the bone region calculated by a different method from the setting of the partial storage region.

### Modification 1

In a case where a partial storage region was edited in accordance with the user's instruction, processing circuitry 11 according to Modification 1 may determine the validity relating to the position and/or size of the edited partial storage region.

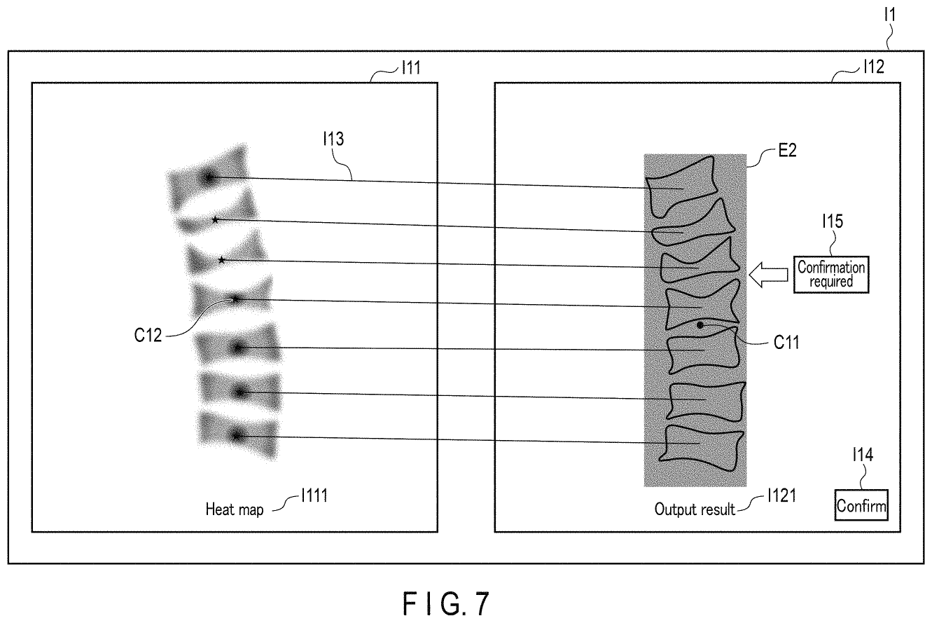

FIG. 7 is a diagram illustrating editing of a partial storage region included in FIG. 7. In accordance with the user's instruction, the processing circuitry 11 edits one partial storage region in such a manner as to include all vertebra regions included in the medical image in the second display area. For example, if the partial storage region is edited, the processing circuitry 11 determines the validity of the position of the partial storage region in step S14, by setting the center point of the edited partial storage region as the first enter point. Further, the processing circuitry 11 determines the validity of the size of the edited partial storage region, based on the center point of the intervertebral disk region and/or the second center point C12. In one example, the processing circuitry 11 determines that the validity of the size of the partial storage region is absent if center points of two or more intervertebral disk regions or two or more second center points are included in the partial storage region, and determines that the validity of the size of the partial storage region is present if one center point of the intervertebral disk region or one second center point is included in the partial storage region. As illustrated in FIG. 7, since the edited partial storage region includes two or more second center points, the validity is determined to be absent. The partial storage region determined to have no validity is displayed with the second visual effect E2, and the character string I15 prompting the user to edit is also displayed.

According to Modification 1, it is possible to determine the validity with respect to the partial storage region that was edited in accordance with the user's instruction, and, by extension, it is possible to reduce the time and labor needed for examining the validity of the partial storage region that was edited in accordance with the user's instruction.

### Modification 2

Processing circuitry 11 according to Modification 2 may move the partial storage region to a position where the validity is determined to be present, if the validity of the position at which the partial storage region is set is determined to be absent. The processing circuitry 11 moves, for example, the center point of the partial storage region, which was determined to have no validity, to the position of the second center point. The processing circuitry 11 may display on the display screen the partial storage region after the movement to the position where the validity is determined to be present.

According to Modification 2, the accuracy of setting of the partial storage region can be improved, without changing the existing algorithm of setting of the partial storage region. Further, the time and labor for the user to edit the partial storage region can be reduced.

### Modification 3

For example, in a case where the partial storage region edited in accordance with the user's instruction is determined to have no validity, processing circuitry 11 according to Modification 3 displays a display screen prompting the user to make confirmation before finally confirming the partial storage region. The partial storage region, which was edited in accordance with the user's instruction, is, for example, one partial storage region including two or more vertebrae. In one example, in FIG. 7, in a case where the button I14 for confirming the partial region is selected, a dialog box including buttons displaying a character string such as “Partial storage region confirmed?” and character strings such as “Yes” and “No” is displayed on the display screen I1. If the partial storage region that is determined to have no validity is confirmed in accordance with the user's instruction, the processing circuitry 11 may display the partial storage region with the first visual effect.

According to Modification 3, the partial storage region that is determined to have no validity can be determined to have validity in accordance with the user's instruction, and, by extension, the determination of a different standpoint from the determination of validity can be executed in accordance with the user's instruction.

### Modification 4

Processing circuitry 11 according to Modification 4 displays, for example, in regard to a partial storage region determined to have no validity, a selectable display part relating to editing of this partial storage region, and, if the display part is selected, the processing circuitry 11 sets an initial position of the partial storage region in the editing, based on the second center point. The number of partial storage regions that are initially disposed is determined by, for example, the number of second center points included in the partial storage region of the target of editing. The size and position of the initially disposed partial storage regions are defined, for example, such that the partial storage region of the target of editing is divided into the number of initially disposed partial storage regions by a plane perpendicular to the center line.

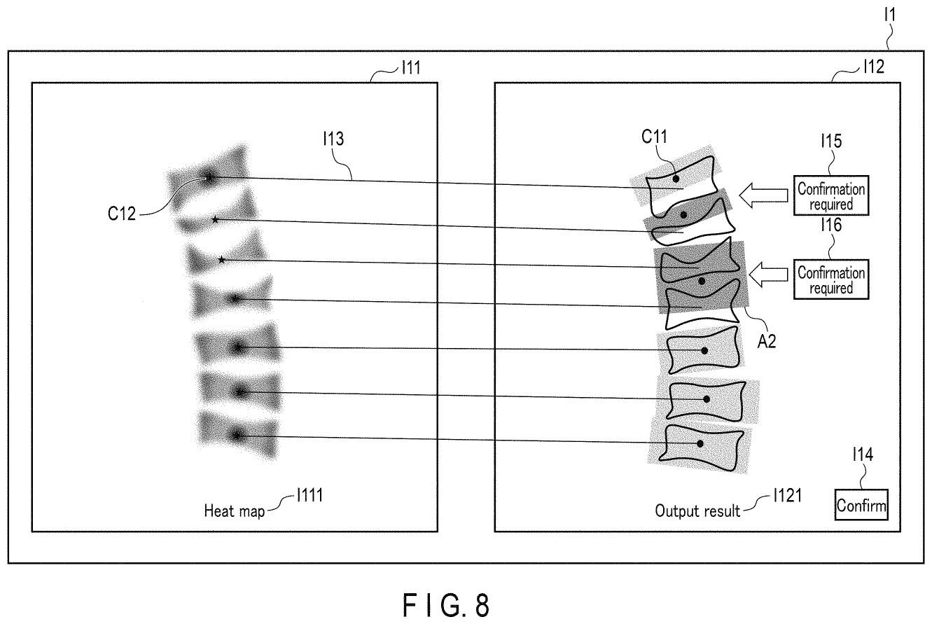

FIG. 8 is a diagram exemplarily illustrating a state before editing of a partial storage region according to Modification 4. Two second center points are included in a partial storage region A2 determined to have no validity. In connection with the partial storage region A2, a display part I16 is also displayed. The display part I16 is, for example, a button on which the character string I15 prompting editing is displayed. If the display part I16 is selected, the editing of the partial storage region A2 is started. The display part I16 is selected in accordance with the user's instruction via the input interface 41.

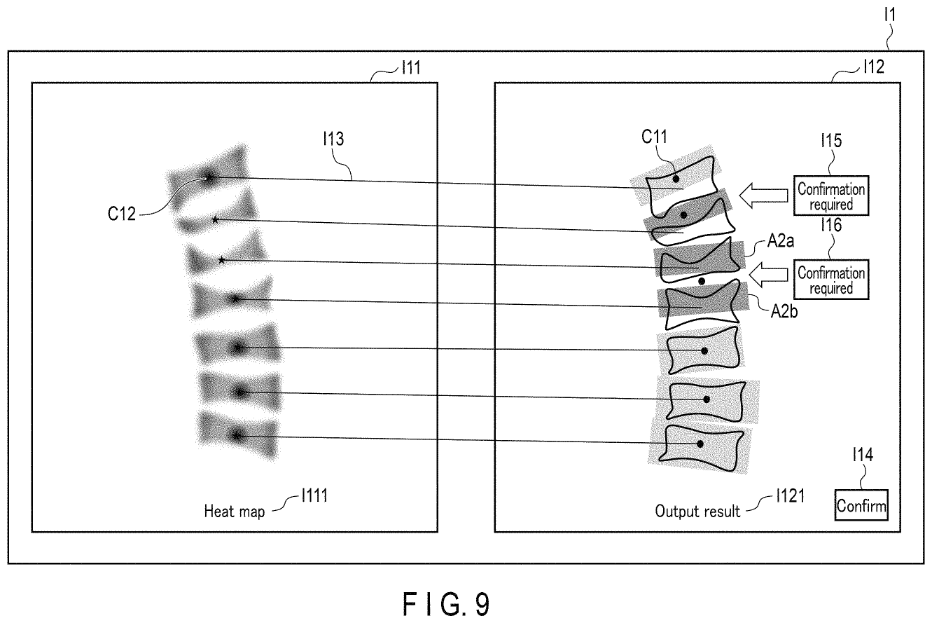

FIG. 9 is a diagram exemplarily illustrating an initial position of the partial storage region according to Modification 4. As illustrated in FIG. 9, instead of the partial storage region A2 in FIG. 8, a partial storage region A2a and a partial storage region A2b are displayed as an initial disposition of the partial storage region to be edited. If the button I16 is selected, the editing of the partial storage region A2 including two second center points is enabled, and the two partial storage regions, namely the partial storage region A2a and the partial storage region A2b, are set as the initial disposition of editing. The initially arranged partial storage region A2a and partial storage region A2b are selectably displayed, respectively, and are edited. Specifically, the partial storage regions selected by an operation including dragging are moved to a freely selected position. If the button I14 for confirming the partial storage regions is selected, the editing of the partial storage region A2a and partial storage region A2b is confirmed.

Note that the modification is not limited to the division of the partial storage region that is the target of editing. For example, in a case where one second center point is included in the partial storage region that is the target of editing, the initial disposition of the partial storage region before editing may be set as the initial disposition in the editing of the partial storage region.

According to Modification 4, the initial position of the partial storage region, which is suitable for the content of editing, is set at the time of starting the editing of the partial storage region determined to have no validity. Thereby, it is possible to reduce the amount of work needed for editing the partial storage region determined to have no validity.

### Second Embodiment

In the first embodiment, the validity of the partial storage region is determined based on the spatial distribution and the partial storage region. In a second embodiment, based on the partial storage region determined to have validity, related information in regard to an anatomical part included in the partial storage region is generated. Hereinafer, a medical image processing apparatus according to the second embodiment is described. Structural elements having the same functions as in the first embodiment are denoted by identical reference signs, and an overlapping description is given only where necessary.

By implementing the related information generation function 117, the processing circuitry 11 generates, based on a partial storage region determined to have validity, related information in regard to an anatomical part included in the partial storage region.

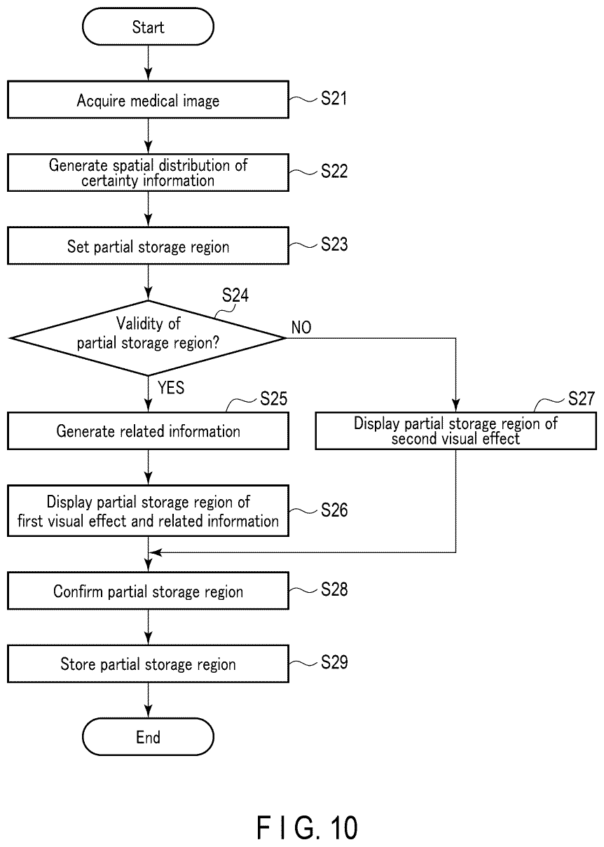

FIG. 10 is a diagram illustrating a processing procedure of a storing process of a medical image according to the second embodiment. As illustrated in FIG. 10, the display process of the display screen according to the second embodiment is a processing procedure in which step S25 is added between step S24 corresponding to step S14 of FIG. 3, and step S26 corresponding to step S15 of FIG. 3. Hereinafter, a process of step S25 onwards is described.

If the validity of the partial storage region is determined to be present in step S24 (step S24: YES), the processing circuitry 11 generates, by implementing the related information generation function 117, the related information that relates to the anatomical part included in the partial storage region, based on the partial storage region determined to have validity (step S25). The related information includes, for example, a label representing a sign or a name of the anatomical part. The processing circuitry 11 generates, on the certainty map, the related information, based on the certainty information of the position included in the partial storage region determined to have validity.

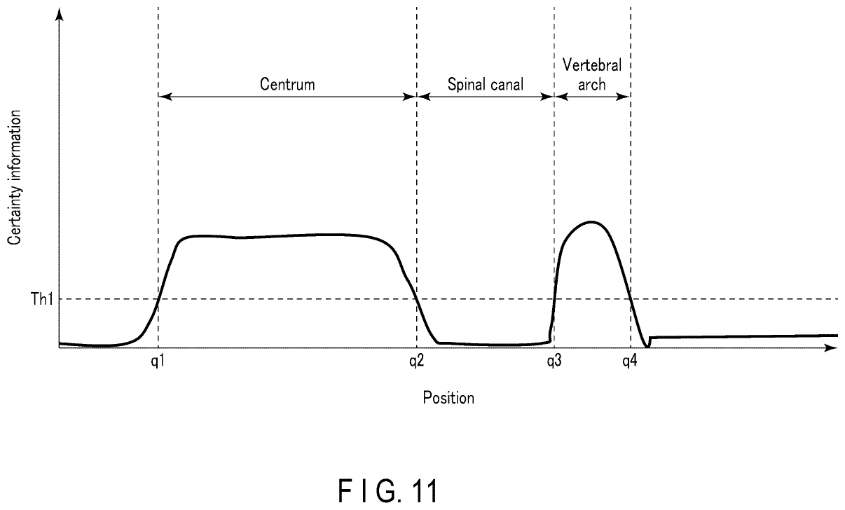

FIG. 11 is a diagram representing certainty information and position information relating to a vertebra. The ordinate axis of the graph of FIG. 11 represents an intensity of certainty information of the bone region. The abscissa axis of the graph represents a position. The graph indicates the certainty information of each of positions in a direction perpendicular to the center line in regard to the partial storage region determined to have validity. In one example, the direction perpendicular to the center line is a direction from the front side to the rear side of the subject. The certainty information may be a value corresponding to a position on a straight line perpendicular to the center line, or may be a value of integration in a finite section in the line direction. A position q1 is a position at which the certainty information exceeds a predetermined value Th1. A position q2 is a position at which the certainty information decreases below the predetermined value Th1. A position q3 is another position at which the certainty information exceeds the predetermined value Th1. A position q4 is another position at which the certainty information decreases below the predetermined value Th1. The processing circuitry 11 generates, by implementing the related information generation function 117, label information of a centrum, a spinal canal, and/or a vertebral arch of the vertebra, based on the length of a section during which the certainty information exceeds the predetermined threshold Th1, and the position of the section. A freely selected value may be set as the predetermined threshold Th1.

As illustrated in FIG. 11, in one example, the processing circuitry 11 segments a set of spatially successive pixels, which exceeds or decreases below a predetermined threshold TH1 between the position q1 and the position q4. A label indicating that the anatomical part is the centrum is added to an image region corresponding to the section between the position q1 and position q2, which is a longest section exceeding the predetermined threshold TH1 among the segmented sections. The processing circuitry 11 adds a label indicating that the anatomical part is the vertebral arch to an image region corresponding to the section between the position q3 and the position q4, which is a section exceeding the predetermined threshold TH1 among the segmented sections and is shorter than the section between the position q1 and the position q2. The processing circuitry 11 adds a label indicating that the anatomical part is the spinal canal to an image region corresponding to the section between the position q2 and the position q3, which is a section interposed between the sections exceeding the predetermined threshold Th1 among the segmented sections and is lower than the predetermined threshold Th1. By labeling the anatomical parts, based on the certainty information and position included in the partial storage region, it is possible to label the centrum, spinal canal and/or vertebral arch in the vertebra.

Note that the labeling of the centrum, spinal canal and/or vertebral arch in the vertebra is not limited to the above-described content. For example, the processing circuitry 11 may label the centrum, spinal canal and vertebral arch, based on a combination of results of a plurality of thresholds. Further, the processing circuitry 11 may label the centrum, spinal canal and vertebral arch in accordance with a distribution of certainty information (for example, the shape of the graph of FIG. 11). Besides, among parts exceeding the predetermined threshold TH1 during a predetermined section, the processing circuitry 11 may label a part located on the front side of the subject as the centrum, and may label a part located on the rear side of the subject as the vertebral arch.

If step S25 is executed, the processing circuitry 11 displays, by implementing the display control function 114, the display screen in which the partial storage region and the related information are superimposed on the medical image (step S26).

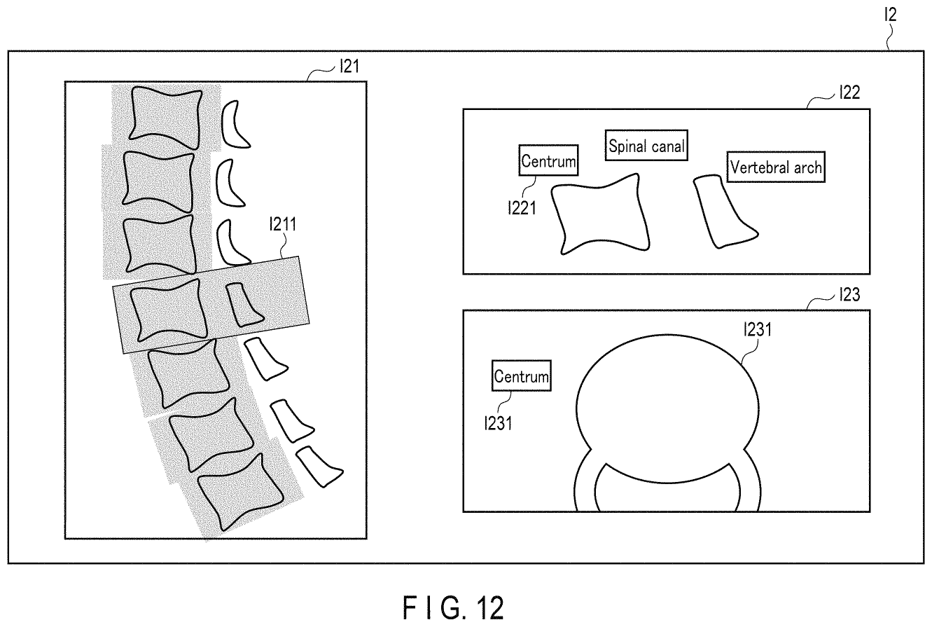

FIG. 12 is a diagram illustrating a display screen I2 displaying the related information. As illustrated in FIG. 12, the display screen I2 includes a first image I21, a second image I22, and a third image I23. The first image I21 is an image in which a partial storage region is superimposed on an oblique cross-sectional image of the spine region. The partial storage region is selectably displayed in accordance with the user's instruction. In one example, it is assumed that a partial storage region I211 of the first image I21 has been selected. The selected partial storage region I211 may be displayed distinguishably from other unselected partial storage regions. For example, the selected partial storage region I211 is displayed by being delineated by a black box line, and box lines are not displayed for the unselected partial storage regions. The second image I22 is an oblique cross-sectional image of the vertebra region included in the partial storage region I211 selected in the first image I21. The labels I221 of anatomical parts generated in step S24 are displayed on the second image I22. As illustrated in FIG. 12, in the labels I221 of anatomical parts, character strings indicating names such as “Centrum”, “Spinal canal” and “Vertebral arch”, are displayed. The third image I23 is an axial cross-sectional image of a region associated with the label I221 of the anatomical part selected in the second image I22. As illustrated in FIG. 11, with the label I221 of the anatomical part of “Centrum” being selected, the third image I23 displays a centrum region I231 in the selected partial storage region I211. In addition, the third image I23 also displays a character string indicating the name of “Centrum” as the label I231 of the anatomical part. By selectably displaying the bone region and the partial storage region, the bone region included in the selected partial storage region can be displayed. In addition, by selectably displaying the bone region and the label together, the region corresponding to the selected label can be displayed.

Note that, as the first image I21, second image I22 and third image I23, images of any cross sections may be displayed. For example, the first image I21 may be an oblique cross-sectional image, the second image I22 may be an axial cross-sectional image, and the third image I23 may be a coronal cross-sectional image. The labeling of anatomical parts is not limited to character strings. For example, the labeling may be signs. Besides, labels of anatomical parts may be displayed on the first image I21. For example, the name of each vertebra may be displayed by being superimposed on the first image I21. Further, aside from the labeling of anatomical parts, morphological information of the bone region, including the presence/absence of fracture of a bone, or a bolt, may be displayed. For example, a character string indicating “presence of a bolt” is displayed on a bone region where a bolt is inserted.

Since step S28 in the second embodiment corresponds to step S17 in the first embodiment, and step S29 in the second embodiment corresponds to step S18 in the first embodiment, a description thereof is omitted.

If step S29 is executed, the storing process of the medical image according to the second embodiment is completed.

According to the second embodiment, it is possible to reduce the time and labor of the user that sets the related information in regard to the anatomical part included in the partial storage region. Moreover, based on the related information, the region to be displayed can be selected in accordance with the user's instruction.

According to at least one of the above-described embodiments, the user's time and labor can be reduced in regard to the setting of the storage region for the medical image.

The term “processor” used in the above description means, for example, a CPU, a GPU, or circuitry such as an application specific integrated circuit (ASIC), a programmable logic device (for example, a simple programmable logic device (SPLD), a complex programmable logic device (CPLD), or a field programmable gate array (FPGA)). The processor implements a function by reading and executing a program stored in storage circuitry. The program may be directly incorporated into the circuitry of the processor instead of being stored in the storage circuitry. In this case, the processor implements the function by reading and executing the program incorporated into the circuitry. On the other hand, in a case where the processor is, for example, an ASIC, the function is directly incorporated as logic circuitry in the circuitry of the processor, instead of the program being stored in the storage circuitry. Each processor of the present embodiment is not necessarily configured as single circuitry, but may include a plurality of units of independent circuitry to implement the functions of the processor. Furthermore, multiple components in FIG. 2 may be integrated into a single processor to implement the functions of the processor.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray computed tomography apparatus comprising processing circuitry configured to:
acquire a medical image including a bone region;
generate a spatial distribution of certainty information of the bone region, based on the medical image;
set a partial storage region for the bone region on the medical image, based on the spatial distribution;
determine validity of the partial storage region, based on the spatial distribution and position information of the partial storage region; and
display a display screen on which the medical image and the partial storage region are superimposed, the displaying including displaying the partial storage region with a first visual effect in a case where the validity is determined to be present, and displaying the partial storage region with a second visual effect different from the first visual effect in a case where the validity is determined to be absent.

2. A medical image processing apparatus comprising processing circuitry configured to:
acquire a medical image including a bone region;
generate a spatial distribution of certainty information of the bone region, based on the medical image;
set a partial storage region for the bone region on the medical image, based on the spatial distribution;
determine validity of the partial storage region, based on the spatial distribution and position information of the partial storage region; and
display a display screen on which the medical image and the partial storage region are superimposed, the displaying including displaying the partial storage region with a first visual effect in a case where the validity is determined to be present, and displaying the partial storage region with a second visual effect different from the first visual effect in a case where the validity is determined to be absent.

3. The medical image processing apparatus of claim 2, wherein the processing circuitry is configured to:
calculate a first center point as a center point of a region in which the certainty information exceeds a first threshold in the spatial distribution; and
set the partial storage region on the medical image, based on the spatial distribution and the first center point.

4. The medical image processing apparatus of claim 3, wherein the processing circuitry is configured to:
calculate a second center point as a center point of a region in which the certainty information exceeds a second threshold less than the first threshold in the spatial distribution; and
calculate a distance between the first center point and the second center point, and determine that the validity is present in a case where a distance from the first center point is less than a third threshold, and that the validity is absent in a case where the distance is not less than the third threshold.

5. The medical image processing apparatus of claim 4, wherein the processing circuitry is configured to:
generate a spatial distribution of certainty information relating to a spine region in the bone region,
calculate the first center point from each of vertebra regions of the spine region, and
calculates the second center point from each of the vertebra regions.

6. The medical image processing apparatus of claim 5, wherein the processing circuitry is configured to:
calculate a center point of an intervertebral disk region in the spine region, and
set a length of the partial storage region in regard to a center-line direction of the spine region, based on a distance between the first center point and the center point of the intervertebral disk region neighboring the vertebral region including the first center point.

7. The medical image processing apparatus of claim 4, wherein the processing circuitry is configured to:
display a selectable display part relating to editing of the partial storage region, in regard to the partial storage region determined to have no validity, and
set, if the display part is selected, an initial disposition of the partial storage region in the editing, based on the second center point.

8. The medical image processing apparatus of claim 2, wherein the processing circuitry is configured to display the partial storage region on the medical image in such a manner that the partial storage region is capable of being edited in accordance with a user's instruction.

9. The medical image processing apparatus of claim 8, wherein the processing circuitry is configured to determine the validity in regard to the partial storage region in a case where the partial storage region is moved.

10. The medical image processing apparatus of claim 9, wherein the processing circuitry is configured to:
confirm a position of the partial storage region in accordance with the user's instruction; and
display, in a case where the partial storage region edited in accordance with the user's instruction is determined to have no validity, a display screen prompting the user to make confirmation before finally confirming the partial storage region.

11. The medical image processing apparatus of claim 2, wherein the processing circuitry is configured to generate related information that relates to an anatomical part included in the partial storage region, based on the partial storage region determined to have validity.

12. The medical image processing apparatus of claim 11, wherein the related information includes, for example, a label representing a sign or a name of the anatomical part.

13. The medical image processing apparatus of claim 11, wherein the processing circuitry is configured to display a display screen on which the related information is further superimposed, in addition to the medical image and the partial storage region.

14. The medical image processing apparatus of claim 11, wherein the processing circuitry is configured to:
calculate a first center point as a center point of a region in which the certainty information exceeds a first threshold in the spatial distribution;
calculate a second center point as a center point of a region in which the certainty information exceeds a second threshold less than the first threshold in the spatial distribution;
generate a spatial distribution of certainty information relating to a spine region in the bone region;
calculate the first center point from each of vertebra regions of the spine region;
calculate the second center point from each of the vertebra regions; and
generate label information of a centrum, a spinal canal, and/or a vertebral arch of the vertebra region by using the certainty information in a direction perpendicular to a center-line direction of the spine region in the partial storage region set in the vertebra region.

15. The medical image processing apparatus of claim 2, wherein the processing circuitry is configured to confirm a position of the partial storage region in accordance with a user's instruction.

16. The medical image processing apparatus of claim 2, wherein the processing circuitry is configured to move the partial storage region to a position where the validity is determined to be present, if the validity is determined to be absent.

17. The medical image processing apparatus of claim 2, wherein the processing circuitry is configured to cut out and store partial data of the medical image, the partial data corresponding to the partial storage region.

18. A medical image processing method comprising causing a computer to execute:
acquiring a medical image including a bone region;
generating a spatial distribution of certainty information of the bone region, based on the medical image;
setting a partial storage region for the bone region on the medical image, based on the spatial distribution;
determining validity of the partial storage region, based on the spatial distribution and position information of the partial storage region; and
displaying a display screen on which the medical image and the partial storage region are superimposed, the displaying including displaying the partial storage region with a first visual effect in a case where the validity is determined to be present, and displaying the partial storage region with a second visual effect different from the first visual effect in a case where the validity is determined to be absent.

19. A non-transitory computer readable medium including computer executable instructions, wherein the instructions, when executed by a processor, cause the processor to perform operations comprising:
acquire a medical image including a bone region;
generate a spatial distribution of certainty information of the bone region, based on the medical image;
set a partial storage region for the bone region on the medical image, based on the spatial distribution;
determine validity of the partial storage region, based on the spatial distribution and position information of the partial storage region; and
display a display screen on which the medical image and the partial storage region are superimposed, the displaying including displaying the partial storage region with a first visual effect in a case where the validity is determined to be present, and displaying the partial storage region with a second visual effect different from the first visual effect in a case where the validity is determined to be absent.
